(19) United States
(12) Patent Application Publication
(10) Pub. No.: US 2007/0055225 A1
(43) Pub. Date: Mar. 8, 2007

(54) Method and apparatus for electromagnetic ablation of biological tissue

(76) Inventors: Gerald D. Dodd III, San Antonio, TX (US); Rex D. Boykin, San Antonio, TX (US); Hayden Head, San Antonio, TX (US)

Correspondence Address: TROP PRUNER & HU, PC, 1616 S. VOSS ROAD, SUITE 750, HOUSTON, TX, 77057-2631, US

(21) Appl. No.: 11/514,592

(22) Filed: Sep. 1, 2006

Related U.S. Application Data
(60) Provisional application No. 60/714,808, filed on Sep. 7, 2005.

(51) Int. Cl. A61B 18/18

(52) U.S. Cl. 606/34; 606/32

(57) ABSTRACT

Apparatus and methods suitable for electromagnetic ablation of targeted tissue provide for improved control over ablation size and shape. In various embodiments, a plurality of dispersive electrodes may be arranged in a circumferential manner in close proximity to a targeted tissue region. The dispersive electrodes may be individually controlled to alternately enable/disable the electrodes to provide for different current paths between an active electrode and one or more of the dispersive electrodes. Other embodiments are described and claimed.

This application claims priority to United States Provisional Patent Application No. 60/714,808 filed on Sep. 7, 2005 in the name of Gerald D. Dodd, III, Rex D. Boykin and Hayden Head, entitled METHOD AND APPARATUS FOR ELECTROMAGNETIC ABLATION OF BIOLOGICAL TISSUE.

### FIELD OF THE INVENTION

Embodiments of the present invention relate to electrosurgery, and more particularly to ablation of tissue via electromagnetic energy such as radio frequency (RF) or microwave signals.

### BACKGROUND

Electrical energy can be used in various surgical modalities to effect a desired result on a patient. For example, high-frequency electrical signals can be applied percutaneously to destroy diseased tissue, such as tumors, cancerous tissue, abnormal cellular formations, and the like. As examples, ablations may be performed using radio frequency signals (i.e., RF ablation) or using microwave energy (i.e., microwave ablation). These surgical modalities are typically performed using a needle inserted locally to the targeted tissue. The needle includes one or more electrodes to transmit an electrical current to the tissue, which heats the tissue, often to a temperature greater than about 50° C. to destroy the targeted tissue.

Different types of ablation systems are used including so-called monopolar ablation and bipolar ablation. Generally, monopolar ablation uses a needle having a single electrode that transmits current to the targeted tissue. Furthermore, this current is dispersed through the body and routed to a low impedance-grounding pad, often referred to as a return or dispersive electrode that is typically placed at a location far removed from the targeted tissue. By placing the return electrode at a location quite remote to the targeted tissue, the return current is dispersed over a large area in the body, as well as over a large surface area of the dispersive electrode, improving safety as undesired heating and/or burning of skin or other tissue in close contact with the dispersive electrode is avoided.

In other implementations, multiple needles may be inserted around a targeted tissue, and the needles may be controlled to emit electric current such that the targeted tissue is ablated and a return current path between the electrodes and distant dispersive electrodes is afforded. In bipolar ablation, a needle inserted includes both an active electrode that transmits electric current and a ground electrode that forms a path for the current. In this manner, targeted tissue located between the electrodes can be effectively ablated.

Dispersive electrodes used are typically large and are placed over areas of a patient that are far removed from the targeted tissue. Furthermore, these electrodes are typically located over areas having significant muscle mass, such as a patient's thighs. It is believed that in so doing a wider dispersive area for the return current is provided, which reduces the likelihood of undesired heating or burning of non-targeted areas of a patient.

While these ablation techniques are commonly used, they are often only effective for targeted tissue of a relatively small size, for example, a few cubic centimeters of tissue at most. Furthermore, the direction and shape of ablation growth cannot be well controlled. Instead, ablation typically occurs radially outwardly in all directions from an active electrode. Thus the lesion formed is typically of a spherical shape and a relatively small size.

To attempt to perform ablations on larger sized tissue, a needle may be inserted in one location and ablation performed, and then the needle may be moved to a close, but different location for further ablation. However performing ablation in this manner complicates the procedure and often results in remaining untreated or unablated targeted tissue.

A need thus exists for improved electromagnetic ablation.

### SUMMARY OF THE INVENTION

In one embodiment, the present invention includes a method for performing electromagnetic ablation. The method may include locating multiple return electrodes on a patient, inserting a single active electrode into the patient, and supplying a current to the active electrode and individually controlling the return electrodes to provide alternating return paths between the active electrode and selected return electrodes. The individual control may thus effect tissue ablation via current flow between the active electrode and at least some of the return electrodes according to selective enabling of the return electrodes, e.g., sequential enabling. The return electrodes may be controlled in a variety of manners, e.g., individually, to provide greater control over size, shape, and direction of a lesion.

More so, the return electrodes can be arrayed on a patient in different ways depending upon a location of the tissue to be ablated. As an example, the return electrodes can be supported by a support structure such as a belt or other radial member for adaptation about a patient. The belt may include a radial member for adaptation about the patient and the return electrodes may in turn be coupled to an interior portion of the radial member. The belt may further include a port to allow for insertion of an active electrode. In such manner, the belt may be located in close proximity to an ablation site, such as adapted around a midsection of the patient. Further, the return electrodes can be controlled by a controller that individually controls the return electrodes to effect the tissue ablation.

Other aspects of the present invention are directed to additional features of such a support structure. These features may include arrangement of the return electrodes in opposing pairs within a radial belt, and one or more sensors to monitor operating conditions of the return electrodes. The support structure may further include a conductor coupled to each return electrode to provide a signal to selectively enable and disable the corresponding return electrode. In some implementations, the return electrodes may be removably coupled to the support structure.

Another aspect of the present invention is directed to a system including a radial belt having return electrodes for positioning on a patient undergoing tissue ablation, and a controller to individually control the return electrodes to effect the tissue ablation. The return electrodes may be individually controllable to provide a return current path for an active electrode such as a monopolar electrode inserted into the patient. In some implementations, the radial belt may be sized to be positioned around the patient's midsection. In some implementations, the controller may activate each of the return electrodes separately, e.g., according to a predetermined treatment plan. The controller may also receive user input during the tissue ablation to modify the predetermined treatment plan.

Applications of embodiments of the present invention in electromagnetic ablation are varied and may be used to effect ablation of many different tissue types.

### BRIEF DESCRIPTION OF THE DRAWINGS

FIG. 1 is a flow diagram of a method in accordance with one embodiment of the present invention.

FIG. 2 is a cross-sectional view of a radial belt in accordance with one embodiment of the present invention.

FIG. 3 is a clinical implementation according to an embodiment of the present invention.

FIG. 4 is a clinical implementation according to another embodiment of the present invention.

### DETAILED DESCRIPTION

In various embodiments, ablation may be performed in a manner that enables improved control over a size and shape of the ablation. To effect such control, embodiments may control a plurality of dispersive electrodes in different manners, depending upon a desired form of the ablation. For example, the multiple dispersive electrodes may be alternately activated such that different current return paths are formed between an active electrode and a selected one or more of the dispersive electrodes. Alternately, multiple dispersive electrodes may be activated at a single time. For instance, opposing electrodes arrayed in a radial fashion may be activated simultaneously, allowing growth of ablation in an outwardly direction towards both activated dispersive electrodes.

In various embodiments, a belt or other support structure may be used to house or support the multiple dispersive electrodes. As an example, a radial belt may include a plurality of individually controllable dispersive electrodes to perform an ablation having a desired shape and size. As described above, the ablation size and shape may be controlled by directing the return current flow between different ones of the dispersive electrodes. In some implementations, a belt may include further components, such as a port to receive the RF needle. Still further, a belt may include temperature or other monitors to be associated with one or more of the dispersive electrodes. Such monitors may provide feedback information to a controller for the dispersive electrodes. In such manner, if one of the dispersive electrodes were to reach a certain temperature, the controller may disable that electrode to prevent harmful side effects, such as heating and burning. Additionally, the belt may include wires and other signal lines to control the dispersive electrodes under control of the controller.

A controller to control ablation in accordance with an embodiment of the present invention may take various forms. For example, a commercially available RF generator may be used that includes additional support for the dispersive electrode control described herein. Furthermore, the generator or a separate control unit may include a switching network to switch the different dispersive electrodes on and off as desired for a particular ablation procedure. The controller may be implemented using various circuitry, logic, or other combinations of hardware, firmware and software to enable directed control of the dispersive electrodes. As an example, a treatment plan for a particular procedure may be provided as an input to a software routine of the controller to cause the different electrodes to be activated as determined in the treatment plan. The controller may also be manipulated dynamically during an ablation procedure to change shape and size of an ablation under process. For example, via a concurrent imaging process, it may be determined that ablation should expand in a particular direction. Accordingly, one or more dispersive electrodes associated with that direction may be enabled to allow ablation expansion in the desired direction.

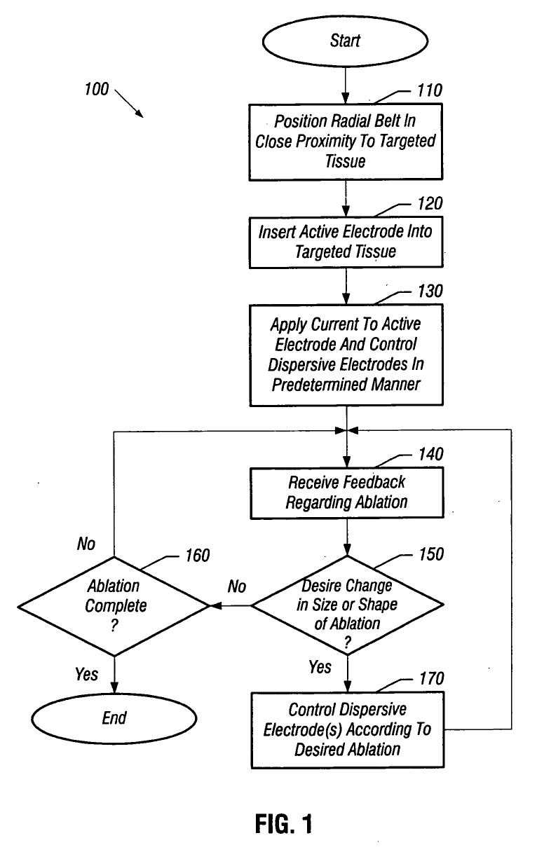

Referring now to FIG. 1, shown is flow diagram of a method in accordance with one embodiment of the present invention. As shown in FIG. 1, method 100 may be used to perform ablation via controlling of dispersive electrodes in accordance with an embodiment of the present invention. As shown in FIG. 1, method 100 may begin by positioning a radial belt in close proximity to the targeted tissue (block 110). While described as using a radial belt, it is to be understood that the scope of the present invention is not so limited and in other embodiments multiple dispersive electrodes may be individually located without use of a belt. For example, instead of the belt, another form of support for the electrodes is possible. Examples may include electrode pads having an adhesive for application directly to the patient. Also, a robotic arm or other support structure may be adapted to retain the dispersive electrodes in a desired manner, e.g., around a patient. However, use of a belt may provide for ease of placement. The belt may be placed in close proximity to the targeted tissue. In other words, the belt may be positioned circumferentially around a patient close to an insertion site for the needle electrode (and even over the insertion site if a port is present) and thus close to the targeted tissue. For example, for ablation of liver or kidney tumors, a belt may be located about a patient's mid section and close to the needle insertion site. For ablation of lung material, the belt may be adapted around the patient's thoracic section. Thus the belt can be located around an area without significant muscle mass. Because in various embodiments dispersive electrode placement is close to the ablation site, the dispersive electrodes may be smaller than typically used grounding pads. In different embodiments, a different number of return electrodes may be present. For example six or more return electrodes may be present in some embodiments while a lesser number of electrodes may be present for use in extremities or other non-core locations. Additionally, smaller and fewer numbers of electrodes may be present for embodiments used in pediatric applications.

Still referring to FIG. 1, next the active electrode may be inserted into the targeted tissue (block 120). The active electrode may be part of a needle assembly and may take many different forms as desired by a practitioner for ablation of particular tissue. In some embodiments, the needle electrode may be inserted through an insertion port within the radial belt. Still referring to FIG. 1, ablation may begin by applying a current to the active electrode while controlling the dispersive electrodes in a predetermined manner (block 130). Thus the dispersive electrodes may be controlled according to, for example, a treatment plan in which different dispersive electrodes are selected for activation at different times during the ablation procedure. Different manners of individually controlling the dispersive electrodes are contemplated and are within the scope of the present invention. As examples, one or more electrodes may be activated at a given time, electrodes may be alternately activated, opposing electrodes may be activated simultaneously and so forth. Thus although described as individually controlling the dispersive electrodes, in various procedures multiple dispersive electrodes may be enabled at a single time, while still being individually controlled. In some embodiments, direction of return current flow between the active electrode and the dispersive electrodes can determine the direction of growth of the ablation. The size of the ablation may in turn be controlled by alternating the current path through the multiple dispersive electrodes. In such manner, simultaneous control of both shape and size of the ablation is possible.

During the ablation procedure, various feedback mechanisms may be enabled. Accordingly, feedback regarding the ablation may be received (block 140). For example, the ablation procedure may be performed while also performing an imaging procedure such as ultrasound, magnetic resonance imaging (MRI), or another imaging procedure. Furthermore, electrical feedback regarding the ablation procedure may also be received. Such feedback may include current, power, and impedance levels. Temperature information, such as obtained from temperature sensors associated with each of the dispersive electrodes, may also be received.

Based on at least some of this information it may be determined whether a change in the size or shape of the ablation is desired (diamond 150). For example, based on imaging feedback it may be determined that the targeted tissue has not been fully ablated and ablation should continue in a particular direction or a size of the ablation should be increased. Accordingly, one or more of the dispersive electrodes can be controlled to achieve the desired ablation (block 170). From block 170, control passes back to block 140 for receiving further feedback regarding the ablation.

If instead at diamond 150 it is determined that no change in the ablation is desired, control passes to diamond 160. There it may be determined whether ablation is complete. If so, method 100 ends. If instead it is determined that ablation is not completed, control passes back to block 140 discussed above. While described with this particular implementation in the embodiment of FIG. 1, other methods of controlling dispersive electrodes in accordance with an embodiment of the present invention may be effected.

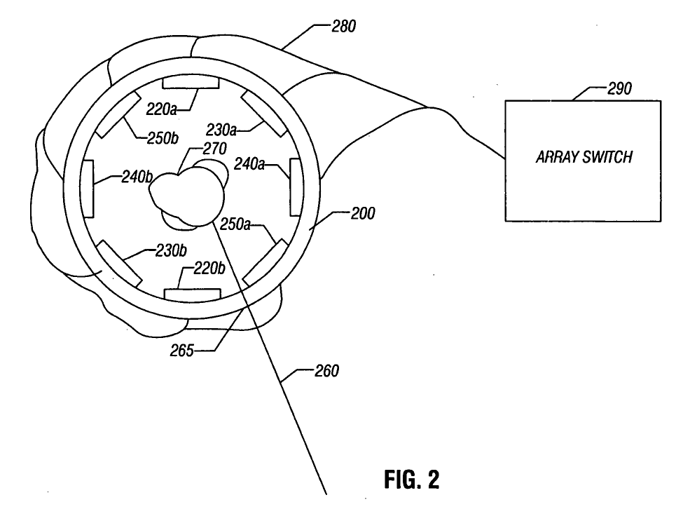

Referring now to FIG. 2, shown is a cross-sectional view of a belt 200 in accordance with one embodiment of the present invention. As shown in FIG. 2, belt 200 may be a radial belt including a plurality of dispersive electrodes. Specifically, as shown in FIG. 2, a plurality of opposing dispersive electrode pairs are present, including opposing electrode pairs 220a and 220b, 230a and 230b, 240a and 240b, and 250a and 250b. While shown with four such opposing pairs, other embodiments may include more or fewer such electrodes and furthermore, the electrodes need not be in opposition to another electrode. As further shown in FIG. 2, an insertion port 265 allows an active electrode 260, e.g., a needle electrode, to be inserted into a targeted tissue 270. By controlling selected ones of the dispersive electrodes, the size and shape of the ablated tissue may be controlled. As further shown in FIG. 2, a plurality of wires 280 are coupled to each of the dispersive electrodes. Wires 280 may provide control signals to activate/de-activate each of the dispersive electrodes. Furthermore, wires 280 may provide a path for feedback information for different sensors associated with the dispersive electrodes (not shown in FIG. 2), and in one embodiment may be controlled by an array switch 290. In many implementations, array switch 290 may be a switch assembly located between a RF generator and belt 200.

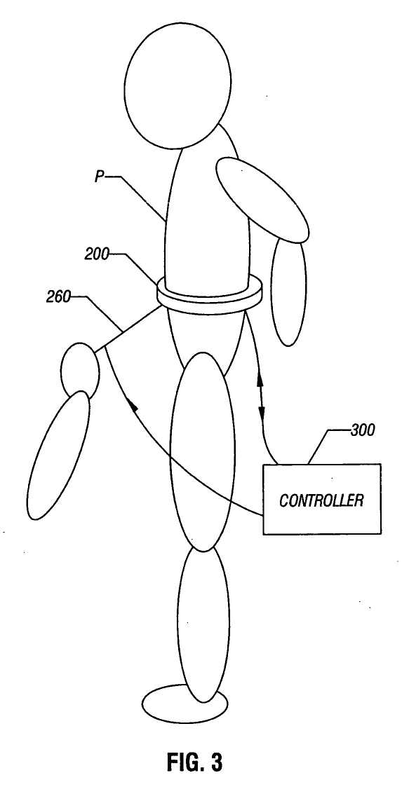

Referring now to FIG. 3, shown is a clinical implementation according to an embodiment of the present invention. As shown in FIG. 3, a patient, P, is lying supine in a surgical environment. A belt 200 in accordance with one embodiment of the present invention is adapted around the patient's midsection. A needle electrode 260 is inserted, either via an insertion port of belt 200, or in close proximity to belt 200, into the patient and more specifically into the targeted tissue or in close proximity thereto.

To perform the ablation, a controller 300, which may be a RF generator, is used to provide a current source to needle electrode 260. Controller 300 further provides control signals to selected electrodes of belt 200 for activation/de-activation of the electrodes according to a predetermined scheme. During ablation, controller 300 may also receive feedback information from belt 200 that can be used to enable dynamic control of the return electrodes, as well as dynamic control of the current source. Thus in this embodiment, controller 300 may include a switching network or other logic to handle the activation/de-activation of the dispersive electrodes selectively.

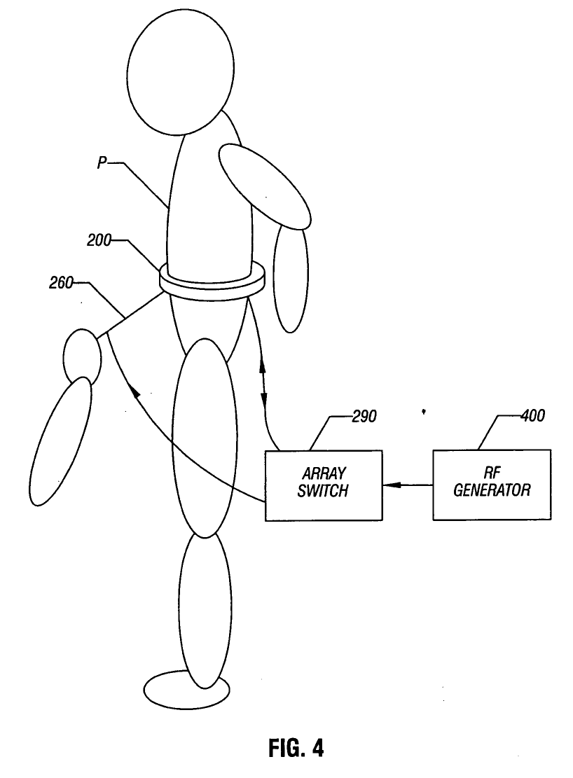

However in other embodiments, an RF generator, such as a conventional RF generator, may be used in connection with an array switch or other switching network or logic to handle the control of individual electrodes of an array in accordance with an embodiment of the present invention. Thus referring now to FIG. 4, shown is a clinical implementation according to another embodiment of the present invention. FIG. 4 is generally consistent with FIG. 3, discussed above. However, instead of a controller 300, an RF generator 400 is coupled to an array switch 290, which in turn is coupled to the individual dispersive electrodes of belt 200. For example, RF generator 400 may be a conventional generator to provide a current source for RF ablation. The RF signals are in turn coupled to array switch 290 which is used to provide the RF signals to selected ones of individual electrodes within belt 200. Of course, other implementations are possible.

Using embodiments of the present invention, ablation may be effected in a desired shape and size. In many embodiments, ablations substantially larger than those possible using conventional ablation may be achieved. For example, ablations may be performed to destroy tumors or other tissue having a volume larger than possible with conventional ablations. Furthermore, eccentric lesions including non-spherical ablations such as oblong or other complex shapes may be effected. Thus embodiments of the present invention provide for directional control of an ablation. While not limited in this regard, ablations and the treatment of different human solid tumors may be effected, including such tumors of the liver, kidney and lungs, for example. Furthermore, by tailoring the shape and size of an ablation zone, tumors or other tissue may be treated with greater accuracy and precision. Likewise, the sparing of surrounding non-targeted tissue and organs can be improved.

While the present invention has been described with respect to a limited number of embodiments, those skilled in the art will appreciate numerous modifications and variations therefrom. It is intended that the appended claims cover all such modifications and variations as fall within the true spirit and scope of this present invention.

What is claimed is:

1. A method comprising:
locating a plurality of return electrodes on a patient;
inserting a single active electrode into the patient; and
supplying a current to the active electrode and individually controlling the plurality of return electrodes to provide alternating return paths between the active electrode and selected ones of the plurality of return electrodes.

2. The method of claim 1, wherein locating the plurality of return electrodes comprises adapting a radial belt including the plurality of return electrodes around a portion of the patient.

3. The method of claim 2, further comprising adapting the radial belt around a midsection of the patient.

4. The method of claim 1, further comprising individually controlling the plurality of return electrodes to control a size of an ablation zone caused by the supplied current.

5. The method of claim 1, further comprising individually controlling the plurality of return electrodes to control a direction of an ablation.

6. The method of claim 1, further comprising individually controlling the plurality of return electrodes to simultaneously control a shape and size of an ablation.

7. The method of claim 1, further comprising individually controlling the plurality of return electrodes according to information from an imaging procedure performed while supplying the current.

8. An apparatus comprising:
a support structure to support a plurality of return electrodes, the support structure to be adapted about a patient during electromagnetic ablation.

9. The apparatus of claim 8, wherein the support structure further comprises an active electrode port to provide for passage of an active electrode through the support structure and into the patient.

10. The apparatus of claim 8, wherein the support structure comprises a radial belt sized to be positioned around a midsection of the patient.

11. The apparatus of claim 10, wherein the plurality of return electrodes are arranged in opposing pairs within the radial belt.

12. The apparatus of claim 8, further comprising a conductor coupled to each of the plurality of return electrodes, the conductor to provide a signal to selectively enable and disable the corresponding return electrode.

13. The apparatus of claim 8, wherein the plurality of return electrodes are removably coupled to the support structure.

14. A system comprising:
a radial belt to support a plurality of return electrodes for positioning on a patient undergoing tissue ablation, the plurality of return electrodes individually controllable to provide a return current path for an active electrode; and
a controller to individually control the plurality of return electrodes to effect the tissue ablation.

15. The system of claim 14, wherein the radial belt is sized to be positioned around a midsection of the patient.

16. The system of claim 14, wherein the controller is to activate each of the plurality of return electrodes according to a predetermined treatment plan.

17. The system of claim 16, wherein the controller is to receive user input during the tissue ablation to modify the predetermined treatment plan.

18. The system of claim 17, wherein the controller is to control the plurality of return electrodes to simultaneously control a size and shape of the tissue ablation.

19. A belt for use in electromagnetic ablation comprising:
a radial member for adaptation about a portion of a patient; and
a plurality of dispersive electrodes coupled to an interior portion of the radial member, the plurality of dispersive electrodes for placement on the patient.

20. The belt of claim 19, further comprising a plurality of conductors, each to provide a return current path for an associated one of the plurality of dispersive electrodes.

21. The belt of claim 20, further comprising a plurality of sensors coupled to the radial member to monitor information during the electromagnetic ablation.

22. The belt of claim 21, wherein the plurality of conductors are each to further communicate the information to a controller from the belt.

23. The belt of claim 19, further comprising an insertion port in the radial member to allow passage of an active electrode through the radial member and into the patient.

24. The belt of claim 19, wherein the belt is sized to be adapted around a midsection of the patient.
